(19) United States
(12) Patent Application Publication
(10) Pub. No.: US 2006/0213509 A1
(43) Pub. Date: Sep. 28, 2006

(54) Device and method for dispensing a fluid

(76) Inventors: Denis Marin, Bailly (FR); Serge Sonie, La Riviere-Saint-Sauveur (FR)

Correspondence Address: HARNESS, DICKEY & PIERCE, P.L.C., P.O. BOX 828, BLOOMFIELD HILLS, MI, 48303, US

(21) Appl. No.: 11/343,277

(22) Filed: Jan. 30, 2006

Related U.S. Application Data
(63) Continuation of application No. PCT/FR04/02062, filed on Jul. 30, 2004.

(30) Foreign Application Priority Data
Jul. 31, 2003 (FR) 03/09463

(51) Int. Cl. A61M 11/00

(52) U.S. Cl. 128/200.23

(57) ABSTRACT

The invention concerns an automatic fluid dispensing device comprising a container, a push-button, a plurality of springs, at least one cam, a plunger, and an aerosol containing the fluid to be dispensed. The invention is characterized in that it further comprises a sealed pressure chamber provided with at least one outlet for releasing the pressure from the pressure chamber when the latter is higher than atmospheric pressure, the compression of the push-button acting, via the cam of the plunger to increase the pressure in said chamber acting on the aerosol so that it delivers the fluid during a predetermined time. The invention also concerns a method using said device.

### CROSS-REFERENCE TO RELATED APPLICATIONS

This application is a continuation of Ser. No. PCT/FR2004/002062, filed Jul. 30, 2004, which claims priority to French Application No. 03/09463, filed Jul. 31, 2003, both of which are incorporated by reference herein.

### BACKGROUND AND SUMMARY

The present invention relates to the field of devices for delivering fluid comprising an aerosol. The present invention relates more particularly to a device for delivering cold fluid for medical, self-medication, care, comfort or relief usage aimed at acting on any pathologies relating to a cryotherapy and/or cryosurgery treatment, for dermatological, care, comfort, aesthetic, or well-being usage, for all benefits of cold in its physiological and mechanical qualities.

The prior art already knows the patent U.S. Pat. No. 5,098,428, which describes a cryosurgical instrument in contact with a liquefied refrigerating gas contained in a thermally insulated receptacle. When the normal venting of the receptacle to free air is interrupted, the pressure of the liquefied refrigerating gas can be increased in the receptacle by means of a ballonet. This pressure causes the liquefied refrigerating gas to be sprayed through the nozzle, until normal venting is re-established.

It is proposed, in the patent U.S. Pat. No. 6,296,410, a device serving to administer a certain quantity of a liquid refrigerating agent. This device comprises: a receptacle serving to contain the liquid refrigerating agent; a normally closed valve connected to the said receptacle; and an actuation means serving to temporarily open the valve, comprising a positioning means serving to place in position at least part of the administration element, so that the refrigerating element leaving the valve can reach the administration element. According to a first preferred embodiment, the positioning means comprises a chamber which is connected to the valve and in which the refrigerating agent is introduced when the valve is actuated, this chamber being designed to receive at least part of the administration element.

There is also proposed, in the patent EP 0281212, a device for carrying out a therapeutic treatment, such as the removal of verrucas, over part of the skin of a human or animal body by local freezing, this device comprising a spraying receptacle which contains a refrigerating liquid which boils in the range of temperatures from 0° to −50° C., a pressure reducing and closing valve to be actuated with a finger on an end wall of the spraying receptacle, this valve being provided with a feed tube that extends into the liquid phase of the refrigerant and a discharge tube connected at a first end to an outlet of the valve, characterised in that the second end of the discharge tube is provided with a cotton pad which is placed downstream of an outlet orifice of the second end of the tube and around the external surface of the second end, so that the flow of refrigerant towards the outside takes place through the cotton pad when the valve is actuated.

These devices do not make it possible to distribute, or deliver, automatically a given quantity of fluid for a period also determined in advance, in particular according to the therapeutic requirements in the case where the delivery device contains a therapeutic fluid used for example for dermatological treatments (brown spots, verrucas, and/or other dermal infections). This is because, in this case, a cold fluid is delivered over a particular area, but this fluid must not damage the adjoining areas nor be applied for too long a time nor not a long time enough on the area to be treated. But in particular, these treatments assume the use of a cold-conveying medium (pad) coming into contact with the parts to be treated. This medium risks not only impairing, through its composition or through its use, the intrinsic qualities of the cold, in particular moisture, but also conveying, on the parts to be treated, contagions by contact risking causing a renewal of the pathologies supposed to be treated or developing other pathologies by indirect effect.

The present invention sets out to remedy the drawbacks of the prior art posing a fluid delivery device by virtue of which the user cannot vary the duration of exposure on the area to be treated and does not have to determine himself, conventionally by pressure on the head of an aerosol, the time and quantity to be delivered, that is to say, in the case of therapeutic treatments, the duration of exposure. Moreover, this device meets particular safety requirements since a child cannot use it, the push button, by virtue of the spring with which it is provided, equipping the device being adjusted so as to function only when a pressure of at least 3 daN (30 newtons) is applied. Finally, the user can apply only one dose since the push button remains inactive, until it is released, as soon as a first pressure is applied, until the end of the delivery of the predetermined quantity (and time) of fluid. Thus, by virtue of the delivery device, only the part to be treated receives only the cold, without alteration by means of a medium or vehicle, benefiting from all its qualities for the physiology of the body and inherent characteristics.

To do this, the present invention concerns a device for the automatic delivery of fluid, of the type comprising a container provided with a fluid reserve in the form of an aerosol and a system for triggering the release of the fluid from the reserve, characterised in that the system triggering the release of the fluid cooperates with means providing the delivery of a previously determined quantity of fluid, for a previously determined duration, on an area whose extent is determined in advance. Preferably, the triggering system comprises a push button, a plurality of springs, at least one cam and a piston, the said means consisting of a sealed pressure chamber having at least one leakage orifice for releasing the pressure from the pressure chamber when this is greater than atmospheric pressure, the pushing in of the push button acting, via the cam and piston, so as to increase the pressure in the said chamber acting on the aerosol.

According to a first embodiment of the invention, the leakage orifice will consist of an opening with a diameter that is variable, by virtue for example of the tightening/slackening of a screw. According to a second embodiment of the invention, the leakage orifice will have a fixed or invariant diameter. In this case, the diameter of the leakage orifice has been chosen according to the requirements relating to the delivery of the fluid, in terms of quantity delivered and/or delivery time, for example according to a therapeutic treatment. Thus the diameter of this leakage orifice is fixed/defined in advance at the time of the production/manufacture of the delivery device according to the invention.

Advantageously, the device of the invention will comprise an actuator, in contact with the above-mentioned cam, having a plurality of protuberances and able to be brought into the locking position following a pressure exerted on the push button. According to one embodiment of the invention, the cam will have an oblique part able to cause the rotation of the actuator when a pressure is exerted on the push button. In this embodiment, the cam will comprise an oblique portion cooperating with a protuberance on the actuator when pressure is exerted on the push button.

Advantageously, the pressure chamber will comprise at least one spring compressed when the piston is pushed in order to act on the aerosol, which consequently releases fluid. Advantageously, the device of the invention can comprise a stop element able to stop the translation of the actuator. Advantageously, the actuator comprises, at its opposite end, at least one protuberance cooperating with at least two oblique parts of the piston in order to effect the translation of the said piston following the pressure exerted on the push button.

According to a preferred embodiment of the invention, the device comprises a end piece, disposed at one of its ends, for example substantially frustoconical in shape. The end piece will be at least partly transparent and/or perforated so as to facilitate the placing of the said end piece. Advantageously, the aerosol will comprise a valve provided with an internal pocket or a ball able to allow the delivery of the fluid whatever the position of the device.

The present invention also relates to a device for delivering a fluid for medical usage relating to a cryotherapy or cryosurgery treatment, the said device being as designed above, characterised in that the aerosol releases a fluid at a temperature of between −20 and −65 degrees. This device is used for any physiological or mechanical action of cold on dermatological pathologies. Preferably, the fluid will be dimethyl ether or any other cold product acting for a cryotherapy or cryosurgery treatment, for example in order to relieve acute pain or treat a brown spot by burning with cold, and any pathologies in general on which cold has a therapeutic, beneficial, regression or relief effect. Advantageously, the container will consist of an elongate body having a diameter of between 20 and 25 millimetres (mm).

The present invention also relates to a method for the automatic delivery of a fluid for a predetermined period by virtue of a device as defined above, comprising a step of triggering by a user consisting of pressing on a push button, characterised in that it comprises the following steps:

sliding a piston over or in a pressure chamber so as to increase the pressure prevailing in the said chamber,

pressing at least one spring, present in the said pressure chamber,

pressing the said pressure chamber on the aerosol (air+spring),

delivering fluid by means of the aerosol,

leakage of gas contained in the pressure chamber through at least one orifice so as to re-establish a pressure close to atmospheric pressure in the said chamber,

sliding of the piston under the action of the actuator,

rotation of the actuator cancelling out the pressure on the aerosol so that the latter stops delivering fluid.

According to a first embodiment of the invention, the duration of delivery of fluid will be variable by adjusting the diameter of the opening of the leakage orifice. According to a second embodiment of the invention, the duration of delivery of fluid will be invariable by calibration of a leakage orifice. Preferably the method of the invention will comprise a supplementary step of resetting, or releasing, of the device when the pressure in the pressure chamber has returned close to or equivalent to atmospheric pressure. Advantageously, the method according to the invention will comprise a step of locking the device, by virtue of an actuator and a stop element, during which the push button will be inactive. Advantageously, the push button will become active again following the unlocking step.

### BRIEF DESCRIPTION OF THE DRAWINGS

The invention will be understood better with the help of the description, given below purely for explanation, of an embodiment of the invention, with reference to the accompanying figures:

FIG. 1 illustrates a perspective view of the device according to the invention, the container being open and separated into two halves, bottom and top, in order to view the elements present in the device, the aerosol and the pressure chamber being visible by virtue of an oblique section;

FIG. 2 illustrates a partial perspective view of the bottom half of the container without the push button, its spring and the cam;

FIG. 3 illustrates a perspective view of FIG. 2 at the moment when the delivery device delivers or ejects a certain quantity of fluid.

### DETAILED DESCRIPTION

As can be seen in the various accompanying figures, the delivery device according to the invention has an elongate shape, substantially identical to a pen. Thus the choice of the valve turned, in the example chosen to illustrate the invention, to a valve, equipping the aerosol, having a diameter of 20 millimetres, not 25.4 mm. These two diameters represent the two conventional sizes of valve for an aerosol.

The device according to the invention comprises a container 1, external body or envelope, in which there are housed all the elements cited below, and which protects the whole of the mechanism of the device in particular against impacts. The device of the invention comprises two ends, at which there are present respectively a push button 2 and a end piece 3. The push button 2 has a conventional cylindrical shape and houses, in its hollow, or empty, interior a spring 4 and a cam 5; the spring 4 being disposed between the internal face of the push button 2 and the external face of the cam 5. The cam 5 is in two main parts, both cylindrical, the first around which the spring 4 is situated and the second having a diameter greater than the first part and the end of which has an oblique slope, or cut.

The cylindrical protuberance of an actuator 6, complementary in shape or equal to that of the oblique end of the cam 5, is placed in contact against the oblique end of the said cam 5. The cam 5 and the actuator 6 are free to rotate whilst only the piston 7 is free in translation. The actuator 6 comprises a plurality of protuberances 8 disposed on its cylindrical periphery.

A stop element 9 is present between the envelope body 1 and the actuator 6 in order to stop the translation of the actuator 6 and therefore the translation of the piston 7. The piston 7 is in contact with the other end of the actuator 6, this end of the actuator having a protuberance 10 extending perpendicular to the longitudinal direction of the end of the actuator 6. The end of the piston 7 in contact with the end of the actuator 6 has respectively a special section, namely two oblique parts 11, 11′ forming between them substantially a V, that is to say the two oblique parts 11, 11′ have contrary or opposed slopes. At the intersection of these two oblique parts 11, 11′ or slopes, there is a point of immobilisation of the protuberance. This is because, when the actuator 6 turns, it drives in rotation the protuberance 10, which slides on the first slope 11 or oblique part of the end of the piston 7 until it is immobilised before the intersection of the two slopes. When the protuberance 10 engages on the second oblique part, the piston 7 is in the process of retracting, in order to return to its initial position, that is to say its position before pressing the push button 2.

The piston 7 is followed by a pressure chamber 12 in which there is housed a spring 13 joining one of the ends of the piston 7 to the internal face of the end of the aerosol 14. The piston 7, when it advances or progresses rapidly, makes the pressure increase in the said chamber 12. The air contained in this chamber 12 is compressed and its rigidity, or pressure, increases until it becomes greater than the rigidity of the spring contained in the valve 15, the aerosol 14 opens. This is why a leakage orifice, not visible in the accompanying figures, is provided between the pressure chamber 12 and the opposite side of the piston 7.

In the example chosen to illustrate the invention, and according to a possibility offered by the invention, this orifice has a cylindrical shape with a diameter variable by virtue for example of a screw that a user can tighten or slacken in order to increase or decrease the diameter of this orifice, the consequence of which is reducing or increasing the speed of re-establishment of a pressure close to or equivalent to atmospheric pressure inside the pressure chamber 12. Thus, knowing the necessary or desired duration and/or the quantity of fluid to be delivered, for the distribution of the fluid 17, the user will adjust the diameter of the orifice accordingly, knowing that the time of emission or delivery of the fluid 17 is related in a linear manner to the diameter of this outlet orifice.

In another embodiment, the orifice has an invariable diameter, the device not having any tightening/slackening screw for adjusting the diameter of this orifice. Thus the delivery device uses a pneumatic timing based on a leakage of air, carried out in the chamber 12. This chamber is ideally interposed between the piston 7 and the casing of the aerosol 14.

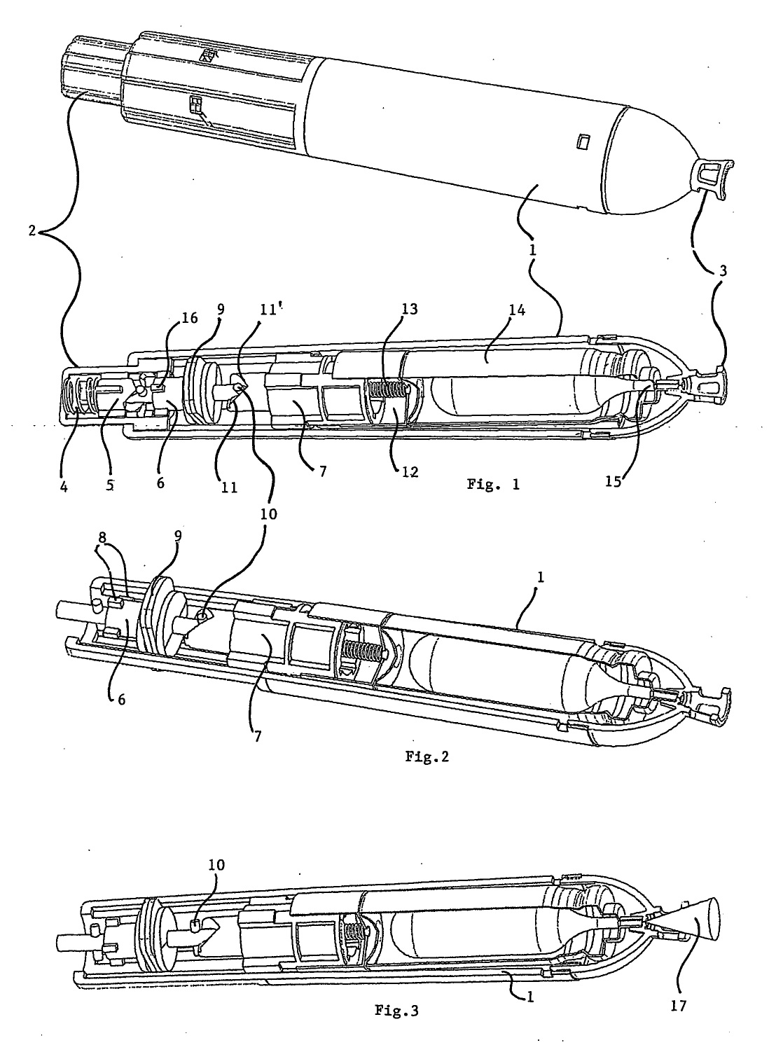

As depicted in particular in FIG. 1, a triggering push button 2 is positioned at the end of the pen-shaped device. In the device of the invention, a user presses in this button 2 until it locks automatically at the end-of-travel position. The “pen” cannot be retriggered before the end of ejection of the gas, the button 2 being locked during this phase. During the phase of pushing in the button 2, nothing happens with regard to the mechanism for opening the aerosol 14.

The delivery method or the kinematics of opening and closing of the aerosol 14 is divided into five phases, respectively the triggering by the user, the opening of the aerosol 14, the timing and closure of the aerosol 14, and the resetting of the system. The user triggers the mechanism by pressing on the push button 2. The spring 4 compresses. The actuator 6 is then locked with respect to rotation by means of four studs 16, present on the circumference of the actuator 6, sliding in grooves. After a few millimetres travel, the studs 16 emerge in a chamber, which releases the rotation of the actuator 6. The user can no longer actuate the push button 2, since the system is locked in this position. The rotation of the actuator 6 causes the translation of the piston 7 by means of a cam 5. The spring 13 is compressed along with the air contained in the pneumatic chamber 12. The assembly 7/12/13 provides the translation and opening of the aerosol 14.

The pneumatic chamber 12 has a calibrated hole which allows a leakage of air and therefore a loss of pressure over time. When the pressure in the chamber 12 is close to atmospheric pressure, the piston 7 advances under the force maintained by the actuator 6. The protuberance on the actuator 6 shifts and is situated in line with the slope 11. Under the action of the spring in the chamber 12, the piston 7 rises again, driving in its rising the actuator 6 in rotation, by means of the slope 11′. The rising of the piston 7 releases the pressure on the aerosol 14. The spring contained in the valve 15 closes off the aerosol 14.

The spring 13 then pushes the piston 7 back to its initial position, causing the rotation of the actuator 6, which then pushes on the cam 5 contained in the push button 2. At the end of rotation of the actuator 6 (abutment at the bottom of the cam 5), the push button 2 rises to its initial position again. The system can be used for a new application. Once the push button 2 is locked, a cam mechanism, compressed by the spring 4 contained in the push button 2, then begins to turn, making the piston 7 advance. This piston 7 pushes on the aerosol 14, which compresses the valve 15 and opens it.

Then there is maintenance, by a mechanical method, of the opening of the aerosol 14 for a given time. The leakage of air calibrated by the outlet orifice, which is formed in the chamber 12, makes the pressure in the system drop slowly. At the end of the given time, the actuator 6, which is still driven in rotation by the cam 5 of the push button 2, manages to make the piston 7 advance. The protuberance on the actuator 6 is in line with the slope 11. Under the action of the spring 13 in the chamber 12, the piston 7 rises again, causing the rotation of the actuator 6. The pressure on the aerosol 14 is then zero.

Under the pressure of the rising of the piston 7, the actuator 6 then pivots, releasing the push button 2 (locked until now). Under the common pressure of a return spring 4 and of the spring of the valve 15 of the aerosol 14, the button 2 resumes its initial position. The system is ready for further use.

For good efficacy of the treatment of the distributed fluid 17, when the latter has therapeutic virtues, it is necessary for the end piece 3 of the aerosol 14 to be situated at a constant distance from the area to be treated. The end piece 3 of the device performs this function of spacer between the skin of the patient and the diffusion end piece. This spacer has an ideally conical shape with an end diameter greater than the verruca to be treated. For better centring of the device on the area to be treated, the end piece 3 is partially transparent and perforated.

The invention is described above by way of example. Naturally a person skilled in the art is in a position to produce different variants of the invention without for all that departing from the scope of the patent.

The invention claimed is:

1. A device for the automatic delivery of fluid, the device comprising a container provided with a fluid reserve in the form of an aerosol and a system for triggering the release of the fluid from the reserve, the system for triggering the release of the fluid providing the delivery of a previously determined quantity of the fluid, for a previously determined period, over an area whose extent is previously determined.

2. A device for the automatic delivery of fluid according to claim 1, wherein the triggering system comprises a push button, a plurality of springs, at least one cam, a piston and a sealed pressure chamber having at least one leakage orifice for releasing the pressure from the pressure chamber when it is greater than atmospheric pressure, the pushing in of the push button acting, via the cam and the piston, so as to increase the pressure in the chamber acting on the aerosol.

3. A delivery device according to claim 2, wherein the leakage orifice includes an opening with a diameter that is variable, by virtue for example of the tightening/slackening of a screw.

4. A delivery device according to claim 2, wherein the leakage orifice has a fixed or invariant diameter.

5. A delivery device according to claim 2, further comprising an actuator, in contact with the cam, having a plurality of protuberances and able to be brought into the locking position following a pressure exerted on the push button.

6. A delivery device according to claim 5, wherein the protuberances on the cam enter orifices present in the above-mentioned container when the actuator is locked.

7. A delivery device according to claim 5, wherein the cam will have an oblique part able to cause the rotation of the actuator when a pressure is exerted on the push button.

8. A delivery device according to claim 7, wherein the cam comprises an oblique portion cooperating with a protuberance on the actuator when pressure is exerted on the push button.

9. A delivery device according to claim 2, wherein the pressure chamber comprises at least one spring compressing when the piston is pushed in order to act on the aerosol, which releases fluid accordingly.

10. A delivery device according to claim 5, further comprising a stop element able to stop the translation of the actuator.

11. A delivery device according to claim 5, wherein the actuator comprises, at its opposite end, at least one protuberance cooperating with at least two oblique parts of the piston in order to effect the translation of the said piston following the pressure exerted on the push button.

12. A delivery device according to claim 1, further comprising an end piece, disposed at one of its end, for example substantially frustoconical in shape.

13. A delivery device according to claim 12, wherein the end piece is at least partly transparent and/or perforated so as to facilitate the placing of the end piece.

14. A delivery device according to claim 1, wherein the aerosol comprises a valve provided with an internal pocket or a ball able to allow the delivery of fluid whatever the position of the device.

15. A device for delivering a fluid for any physiological or mechanical action of cold on dermatological pathologies, the device being in accordance with claim 1, wherein the aerosol comprises a fluid at a temperature of between −20 and −65 degrees.

16. A device for delivering a fluid for a cryotherapy treatment such as the relief of an acute pain or a cryosurgery treatment such as the treatment of brown spots, verrucas and/or other dermal infections according to claim 15, wherein the fluid is dimethyl ether or any other cold product.

17. A device for delivering a fluid for the treatment of verrucas and/or other dermal infections according to claim 15, wherein the container includes an elongate body having a diameter of between 20 and 25 millimetres.

18. A method for the automatic delivery of a fluid for a predetermined period, comprising a step of triggering by a user including pressing on a push button, the method further comprising the following steps:
sliding a piston over or in a pressure chamber so as to increase the pressure prevailing in the chamber;
pressing at least one spring, present in the pressure chamber;
pressing the pressure chamber on the aerosol;
delivering fluid by the aerosol;
leakage of gas contained in the pressure chamber through at least one orifice so as to re-establish a pressure close to atmospheric pressure in the chamber;
sliding of the piston under the action of the actuator; and
rotation of the actuator cancelling out the pressure on the aerosol so that the latter stops delivering fluid.

19. A delivery method according to claim 18, wherein the duration of delivery of fluid is variable by adjusting the diameter of the opening of the leakage orifice.

20. A delivery method according to claim 18, further comprising resetting, or releasing, the device when the pressure in the pressure chamber has returned to close to or equivalent to atmospheric pressure.

21. A delivery method according to claim 18, further comprising locking the device, by virtue of an actuator and a stop element, during which the push button is inactive.

22. A delivery method according to claim 18, wherein the push button becomes active again following the release step.

23. A delivery method according to claim 18, wherein the duration of delivery of fluid is invariable by calibration of a leakage orifice.
